(19) United States
(12) Patent Application Publication
(10) Pub. No.: US 2023/0385442 A1
(43) Pub. Date: Nov. 30, 2023

(54) PATIENT RECORD IDENTIFICATION FOR MEDICAL SYSTEMS WITH UNAUTHENTICATED USERS

(71) Applicant: Cardiac Pacemakers, Inc., St. Paul, MN (US)

(72) Inventor: James Sievert, Bradenton, FL (US)

(21) Appl. No.: 18/200,977

(22) Filed: May 23, 2023

Related U.S. Application Data
(60) Provisional application No. 63/346,458, filed on May 27, 2022.

(51) Int. Cl. G06F 21/62; G06F 21/60; G16H 10/60

(52) U.S. Cl. CPC G06F 21/6245; G06F 21/602; G16H 10/60

(57) ABSTRACT

Embodiments herein relate to medical systems that can accommodate use by unauthenticated users while maintaining security and privacy of patient records. In an embodiment, a medical system is included having a control circuit, a video display, and a user interface, wherein the user interface is controlled by the control circuit and displayed on the video display. The medical system can be configured to permit system use by an unauthenticated user, hash direct patient identifying data along with a salt value using a hash function to generate an identification string and save data regarding operations of the medical system during procedure sessions and procedure timing as linked to identification strings. Other embodiments are also included herein.

This application claims the benefit of U.S. Provisional Application No. 63/346,458, filed May 27, 2022, the content of which is herein incorporated by reference in its entirety.

### FIELD

Embodiments herein relate to medical systems. More specifically, embodiments herein relate to medical systems that can accommodate use by unauthenticated users while maintaining security and privacy of patient records.

### BACKGROUND

Patient care may involve the use of many different types of medical equipment. For example, medical equipment used can include imaging systems (such as MM systems, ultrasound systems, and the like), diagnostic systems (such as endoscopy systems), therapy systems (such as ablation systems, various catheter-based systems), and the like.

In many cases, medical equipment may include controls and/or a user interface where the operator of the equipment can set parameters, provide other inputs, control operation of the equipment, receive outputs and other data, etc. In some cases, the equipment may require an operator to provide credentials and then authenticate the same before providing access for use of the equipment. However, in other cases, the equipment may be configured to provide unauthenticated user access.

### SUMMARY

Embodiments herein relate to medical systems that can accommodate use by unauthenticated users while maintaining security and privacy of patient records. In a first aspect, a medical system can be included having a control circuit, a video display, and a user interface, wherein the user interface can be controlled by the control circuit and displayed on the video display. The medical system can be configured to permit system use by an unauthenticated user, hash direct patient identifying data along with a salt value using a hash function to generate an identification string, and save data regarding operations of the medical system during procedure sessions and procedure timing as linked to identification strings.

In a second aspect, in addition to one or more of the preceding or following aspects, or in the alternative to some aspects, the medical system can be configured to discard the salt value at the end of a procedure session.

In a third aspect, in addition to one or more of the preceding or following aspects, or in the alternative to some aspects, the salt value can include a string of characters derived from a cryptographic random number.

In a fourth aspect, in addition to one or more of the preceding or following aspects, or in the alternative to some aspects, the direct patient identifying data can include a patient ID.

In a fifth aspect, in addition to one or more of the preceding or following aspects, or in the alternative to some aspects, the data regarding operations can include operational parameters.

In a sixth aspect, in addition to one or more of the preceding or following aspects, or in the alternative to some aspects, the hash function can include a password hash function.

In a seventh aspect, in addition to one or more of the preceding or following aspects, or in the alternative to some aspects, the password hash function can include Argon2.

In an eighth aspect, in addition to one or more of the preceding or following aspects, or in the alternative to some aspects, the medical system can include at least one selected from the group consisting of an Mill machine, an endoscopy device, a urological device, an RF generator, and a medical laser device.

In a ninth aspect, in addition to one or more of the preceding or following aspects, or in the alternative to some aspects, the medical system can be configured to receive the direct patient identifying data from the unauthenticated user.

In a tenth aspect, in addition to one or more of the preceding or following aspects, or in the alternative to some aspects, the medical system can be configured to receive inputs from the unauthenticated user regarding desired operations of the medical system.

In an eleventh aspect, in addition to one or more of the preceding or following aspects, or in the alternative to some aspects, the medical system can be configured to present identification strings and procedure time information to a system user to verify the association of a procedure record to a particular patient.

In a twelfth aspect, in addition to one or more of the preceding or following aspects, or in the alternative to some aspects, the medical system prompts the system user to input direct patient identifying data.

In a thirteenth aspect, in addition to one or more of the preceding or following aspects, or in the alternative to some aspects, procedure records verified to be associated with a particular patient can be stored in an electronic medical records system.

In a fourteenth aspect, a method of maintaining procedure data on a medical system open for use by unauthenticated users can be included. The method can include hashing direct patient identifying data along with a salt value using a hash function to generate an identification string and saving data regarding operations of medical system during procedure sessions and procedure timing as linked to identification strings.

In a fifteenth aspect, in addition to one or more of the preceding or following aspects, or in the alternative to some aspects, the method can further include discarding the salt value at the end of a procedure session.

In a sixteenth aspect, in addition to one or more of the preceding or following aspects, or in the alternative to some aspects, the method can further include receiving the direct patient identifying data from an unauthenticated user.

In a seventeenth aspect, in addition to one or more of the preceding or following aspects, or in the alternative to some aspects, the method can further include receiving inputs from an unauthenticated user regarding desired operations of the medical system.

In an eighteenth aspect, in addition to one or more of the preceding or following aspects, or in the alternative to some aspects, the method can further include presenting identification strings and procedure time information to a system user to verify the association of a procedure record to a particular patient.

In a nineteenth aspect, in addition to one or more of the preceding or following aspects, or in the alternative to some aspects, the salt value can include a string of characters derived from a cryptographic random number.

In a twentieth aspect, in addition to one or more of the preceding or following aspects, or in the alternative to some aspects, the direct patient identifying data can include a patient ID.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a medical system in accordance with various embodiments herein.

FIG. 2 is a schematic view of a medical care environment in accordance with various embodiments herein.

FIG. 3 is a schematic view of operations in accordance with various embodiments herein.

FIG. 4 is a schematic view of operations in accordance with various embodiments herein.

FIG. 5 is a schematic view of displayed data in accordance with various embodiments herein.

FIG. 6 is a schematic view of displayed data in accordance with various embodiments herein.

FIG. 7 is a schematic view of operations in accordance with various embodiments herein.

FIG. 8 is a schematic view of operations in accordance with various embodiments herein.

FIG. 9 is a schematic view of operations in accordance with various embodiments herein.

FIG. 10 is a schematic view of a data communication environment in accordance with various embodiments herein.

FIG. 11 is a block diagram of components of a medical system in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

In some cases, it can be desirable to configure medical equipment so that it will provide user access without user authentication. It can also be desirable for medical equipment to generate procedure records regarding what has been performed using the medical equipment and have those records related to a particular patient. However, this can create a challenge in terms of maintaining patient data security and privacy.

Embodiments herein can provide for a secure way of storing data related to procedures performed using medical equipment that is configured to permit system use by unauthenticated users while not compromising patient data security and privacy. In an embodiment, a medical system is included having a control circuit, a video display, and a user interface, wherein the user interface is controlled by the control circuit and displayed on the video display. The medical system can be configured to permit system use by an unauthenticated user, hash direct patient identifying data along with a salt value using a hash function to generate an identification string and save data regarding operations of the medical system during procedure sessions and procedure timing as linked to identification strings.

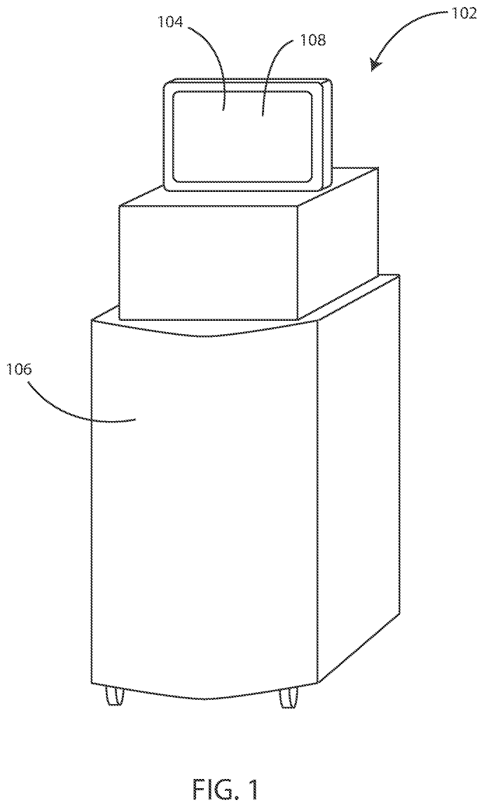

Referring now to FIG. 1, a schematic view of a medical system 102 is shown in accordance with various embodiments herein. The medical system 102 can include a video display 104 and/or can produce data and/or signals to for a video display. The medical system 102 also includes a housing 106. The medical system 102 also includes a user interface 108 that can be controlled by a control circuit and displayed on the video display 104. In various embodiments, the medical system 102 can be an MRI machine, another type of medical imaging or diagnostic machine, an endoscopy device, a urological device, an RF generator, or a medical laser device, amongst others.

In various embodiments, the medical system 102 can be configured to permit system use by an unauthenticated user. For example, the medical system 102 can be configured to a user to interact with the medical system 102 by providing inputs, receiving outputs, and/or controlling the operation thereof without providing a password or other authenticating credential(s)

In various embodiments, the medical system 102 can be configured to hash direct patient identifying data along with a salt value using a hash function to generate an identification string. As such, the system 102 need not and in many embodiments does not store direct patient identifying data, at least while operating in a mode permitting use by non-authenticated users. In various embodiments, the medical system 102 can be configured to save data regarding operations of the medical system 102 related to procedure sessions and procedure timing as linked or correlated to generated identification strings.

In many embodiments, the medical system 102 can be configured to discard a salt value used in a hashing operation after the hashing operation has been performed (such as at the end of a procedure session). With the salt value discarded, the hash output cannot be recreated through brute force techniques even with great effort and therefore there is no practical way to work backwards from the hash output where one way hashing algorithms/functions are used to deduce direct patient identifying data. In this manner, the direct patient identifying data can remain secure.

However, as described below, in some embodiments the salt value may be saved and/or passed to another system or database and saved, such as in a secure system or secure database which an unauthenticated user cannot access. For example, in some embodiments, the salt values can be securely stored and then later securely retrieved to provide verification of the association of particular patients with specific procedure/system records.

In various embodiments, the medical system 102 can be configured to receive direct patient identifying data from an unauthenticated user and/or from another system or device. In various embodiments, the medical system 102 can prompt the system user to input or initiate the input of direct patient identifying data. In various embodiments, the medical system 102 can be configured to receive inputs from an unauthenticated user regarding desired operations of the medical system 102 and/or parameters of the same.

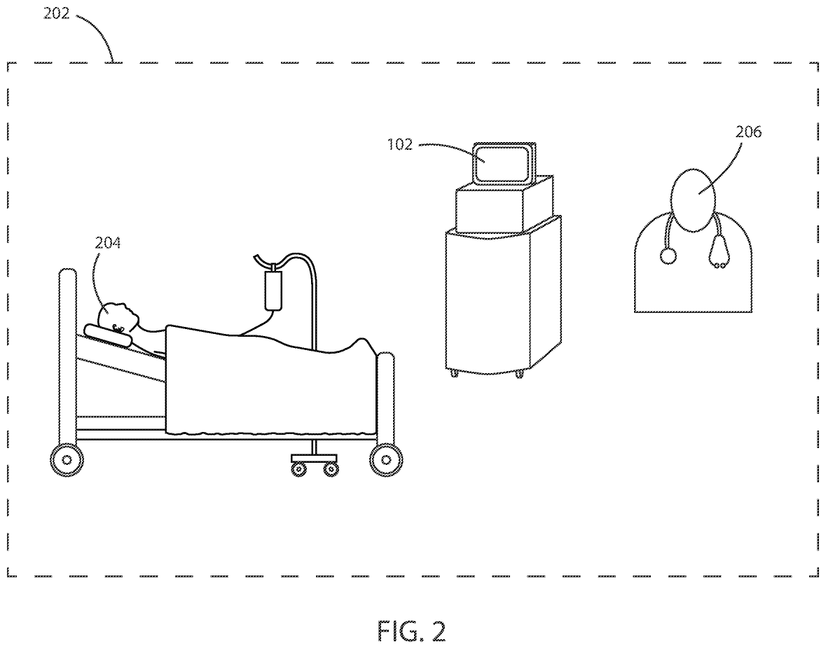

Referring now to FIG. 2, a schematic view of a medical care environment 202 is shown in accordance with various embodiments herein. In specific, FIG. 2 shows a medical system 102 along with a patient 204 and a clinician 206. The clinician 206 can access the medical system 102 without being authenticated. In this manner, desirable and efficient workflows within the medical care environment 202 (which could be an operating room, a procedures room, an imaging lab, or the like) can be maintained. In various embodiments, the medical system 102 can accept user input and/or input from another device or system which can include direct patient identifying data such as a patient ID, social security number, name, or another type of patient identifying data. However, as will be described more fully below, such direct patient identifying data is not directly maintained on the system in many embodiments herein, such as being discarded after execution of a hashing operation, and therefore risks of security and privacy are addressed.

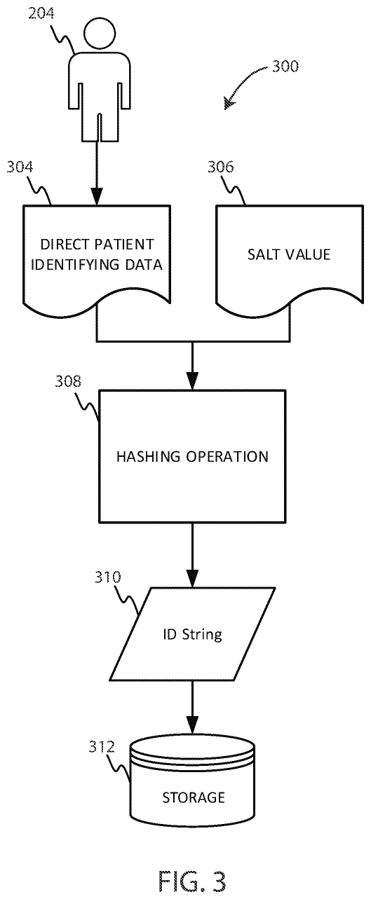

In various embodiments herein, security and privacy can be maintained by only storing an identification string (or tag or procedure identifier), from which direct patient identifying data cannot be derived. Referring now to FIG. 3, a schematic view of operations 300 is shown in accordance with various embodiments herein. Direct patient identifying data 304 may relate to a particular patient 204. A salt value 306 can be used along with the direct patient identifying data 304 as input for a hashing function. The salt value 306 can be generated dynamically or predetermined, such as with a set of predetermined salt values. Generally, hash function outputs can be deterministic, but not when using a salt value 306. The system can take the direct patient identifying data 304 and the salt value 306 and use the two (or a value derived using the two) in a hashing operation 308. The hashing operation 308 can generate an output in the form of an identification string 310 (or tag or procedure identifier) that is unique based on the combination of the direct patient identifying data 304 and the salt value 306. Indeed, since a salt value is used, not only will the identification string 310 be unique across multiple patients, multiple different procedures or records associated with a single patient will end up with unique identification strings 310 (or tags or procedure identifiers).

The identification string 310 can then be stored 312 on the system itself and/or on another device or system separately therefrom and used as described further herein. Specifically, the identification string 310, but not the direct patient identifying data 304 is stored on the system itself. Therefore, the direct patient identifying data 304 can be made secure because the identification string 310 does not allow the direct patient identifying data 304 (or the salt value 306) to be derived therefrom.

Various types of values/data can be used as a salt value. In some embodiments, the salt value 306 can include a string of characters. In some embodiments, the salt value 306 can be a string of random numbers and/or characters. In some embodiments, the salt value can be derived from a cryptographic random number. The length of the salt value can vary. In some embodiments, the salt value can be 1, 2, 3, 4, 5, 6, 7, 8, 9, 10, 12, 15, 25, 30, 40, 50 or more characters in length, or can be a length falling within a range between any of the foregoing.

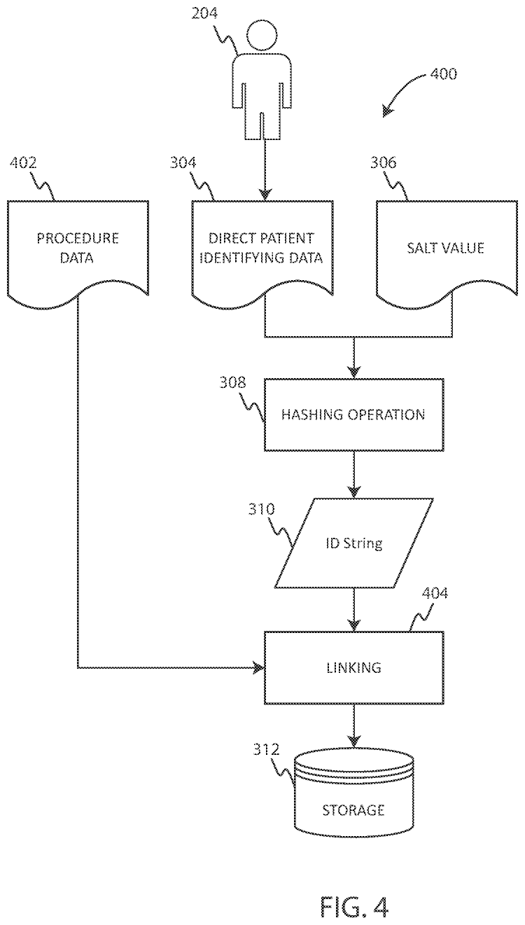

In various embodiments, data related to the procedure performed with the system can be stored along with the generated identification string 310. Referring now to FIG. 4, a schematic view of operations 400 is shown in accordance with various embodiments herein. As before, the system can take the direct patient identifying data 304 and the salt value 306 and process the two in a hashing operation 308. The hashing operation 308 can generate an identification string 310 that is unique based on the combination of the direct patient identifying data 304 and the salt value 306.

Data regarding medical system operations 402 and/or procedures performed therewith can then be linked or correlated with the identification string 310 in a linking operation 404. In some cases, the linkage can be a cross-reference that links an identification string 310 with particular data regarding operations 402. Data regarding operations 402 can include various pieces of information including, but not limited to, procedure date and time, equipment settings during a procedure or session, programs or algorithms used, and the like. The linked identification string 310 and the particular data regarding operations 402 can then be stored 312.

The system can be configured to display data through the user interface (and/or can output the same) including identification string 310 with particular data regarding operations 402. However, as described previously, direct patient identifying data 304 cannot be derived from the identification string 310.

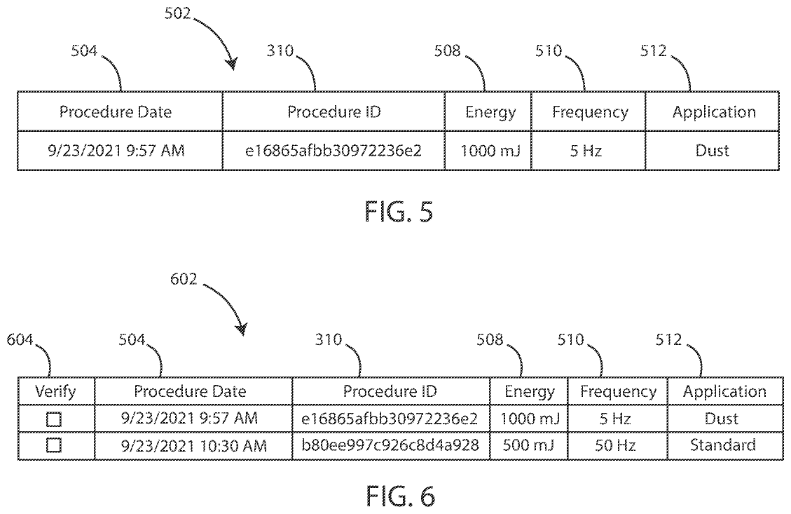

Referring now to FIG. 5, a schematic view of displayed information 502 is shown in accordance with various embodiments herein. The displayed information can include an identification string along with data regarding operations performed (date/time, operational parameters, configuration information, and the like). The displayed information 502 can be provided to a user through the user interface. In this example, FIG. 5 shows a procedure date 504, an identification string 310, an energy 508, a frequency 510, and an application 512. However, various other pieces of data related to a procedure or operations of the medical system can also be stored and displayed.

In some embodiments, the system can be configured to allow a system user to verify aspects of the procedure after the same has been completed. In some embodiments, the system can allow a system user to verify aspects of procedures after a series of procedures have been performed such as at the end of a shift, the end of a day, the end of a week, or the like. In some embodiments, the system can be configured so that the verification takes the form of allowing an authenticated system user to link or correlate records stored on the system such as an identification string along with data regarding operations performed with specific patients, such as for storage within a medical records system.

Referring now to FIG. 6, a schematic view of review information 602 is shown in accordance with various embodiments herein. The review information 602 can be displayed through a user interface. The review information 602 can include a verification options 604 along with data similar to that shown in FIG. 5, such as a procedure date 504, an identification string 310, an energy 508, a frequency 510, and an application 512.

Thus, in various embodiments, the medical system 102 can be configured to present identification strings (such as the output of a hashing operation or derived therefrom) and procedure information (such as procedure time information) to a system user to verify the association or correlation of a procedure record with a particular patient. In various embodiments, the medical system 102 can be configured so that procedure records verified to be associated with a particular patient are stored in an electronic medical records system as linked to the particular patient.

In many embodiments, hash values may be discarded (or otherwise destroyed) after use in a hashing operation so that the particular hashing output cannot be replicated (and thereby the direct patient identifying data cannot be determined, even if selecting from a limited number of possible identities). However, in some scenarios there can be possible value associated with retaining salt values, such as in a secure database and/or remotely from the medical system.

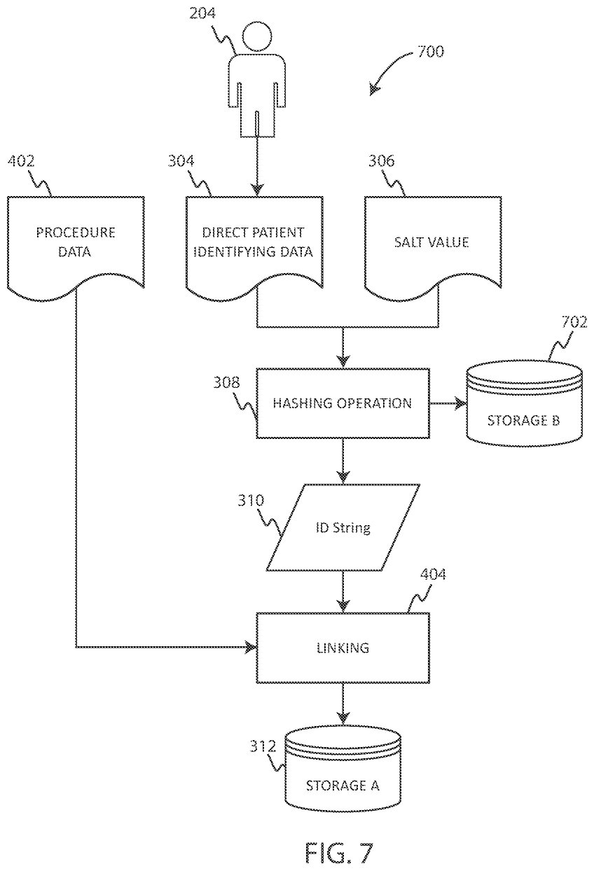

Referring now to FIG. 7, a schematic view of operations 700 is shown in accordance with various embodiments herein. Similar to as described with respect to FIG. 4, the system can take the direct patient identifying data 304 and the salt value 306 and process the two in a hashing operation 308. The hashing operation 308 can generate an identification string 310 that is unique based on the combination of the direct patient identifying data 304 and the salt value 306. Data regarding operations 402 can then be linked with the identification string 310 in a linking operation 404. The linked or correlated identification string 310 and particular data regarding operations 402 can then be stored 312, such as on the system itself (“Storage A”). In this example, however, salt values are not discarded. Rather, the salt values can be stored 702, such as in a secure database (“Storage B”). In some cases, Storage A may be unsecure while Storage B is secure.

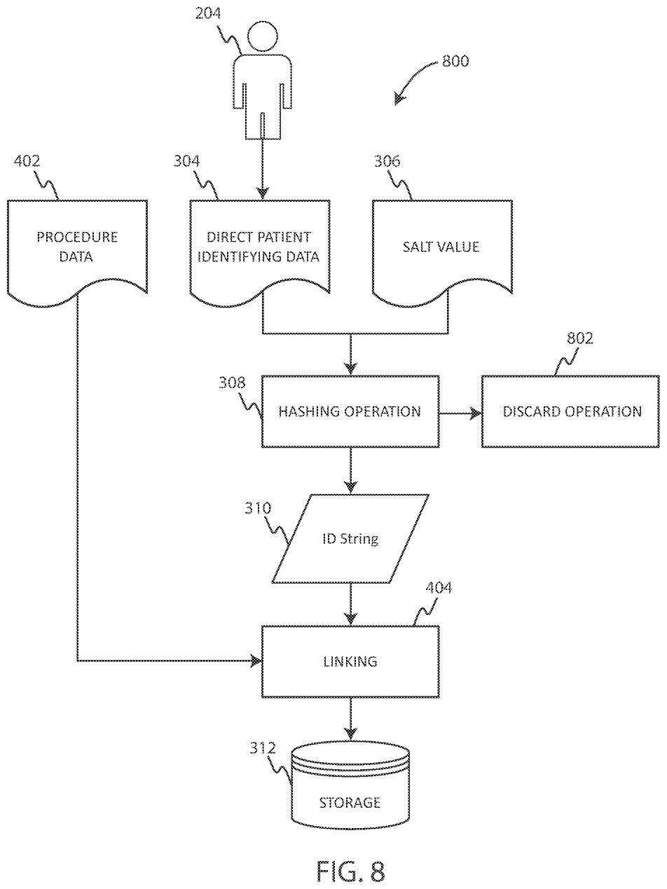

As such, FIG. 7 stands in contrast to FIG. 8. FIG. 8 is a schematic view of operations 800 shown in accordance with various embodiments herein. FIG. 8 is like FIG. 7. However, in the example of FIG. 8, a discard operation 802 is included where both the direct patient identifying data 304 as well as the salt values 306 are discarded. Thus, in various embodiments, the medical system 102 can be configured to discard the salt value 306 at the end of a procedure session.

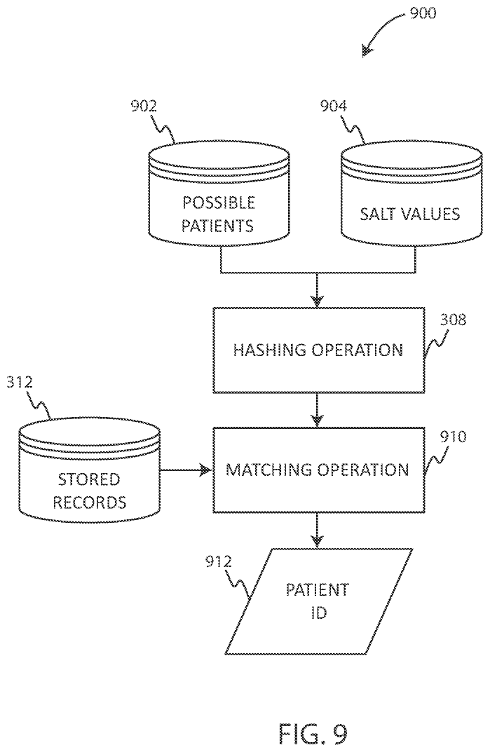

If salt values are retained, then it is possible to execute the hashing operation against a set of possible patients in order to determine which patient is associated with a given identification string 310. For example, in some embodiments, a system can be configured to later use known salt values and a list of possible patients (such as might be attainable from a medical records system) and then execute the hashing operation repeatedly until a hash value is obtained matching a given identification string 310. Referring now to FIG. 9, a schematic view of operations 900 is shown in accordance with various embodiments herein. Inputs can include a set of possible patients 902 along with a set of stored salt values 904. Then, the system can execute hashing operations 308 using different possible patients 902 and different salt values 904 to obtain hashed outputs that might be the same as a given identification string 310 from stored records 312 which can be determined in a matching operation 910. If a match is found, then the system can identify the patient 912 correlated to the given identification string 310.

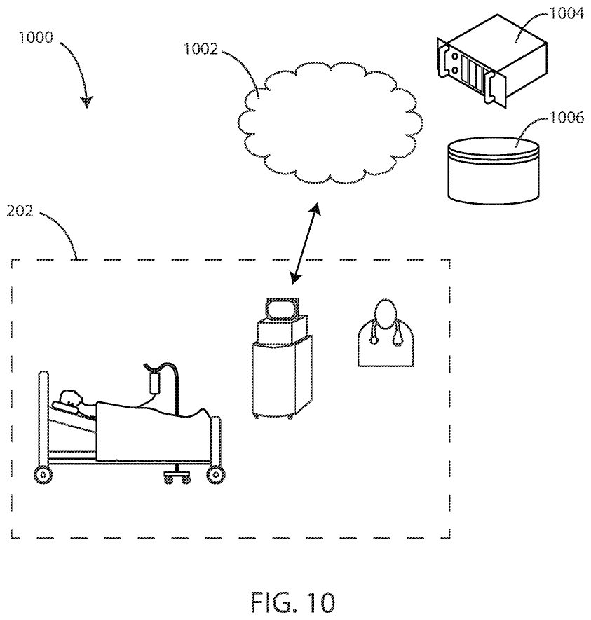

In accordance with embodiments herein, data may be transferred among many different devices/systems as well as passed to different locations (locale, remote, etc.). Referring now to FIG. 10, a schematic view of a data communication environment 1000 is shown in accordance with various embodiments herein. FIG. 10 includes a medical care environment 202, which can include a medical system and can be where the medical system is used by a clinician on a patient. FIG. 10 shows also shows a cloud 1002, which can represent cloud computing resource accessible through a data network. For example, the cloud 1002 can include a server 1004 and a database 1006.

In some embodiments, medical systems herein include a device including a graphical display and a machine-readable medium comprising instructions. The instructions can perform various operations when implemented by one or more processors. By way of example, the operations can include those in accordance with methods or operations as described herein. The machine-readable medium can include random access memory (RAM), read-only memory (ROM), magnetic data storage media, optical data storage media, flash memory, and the like.

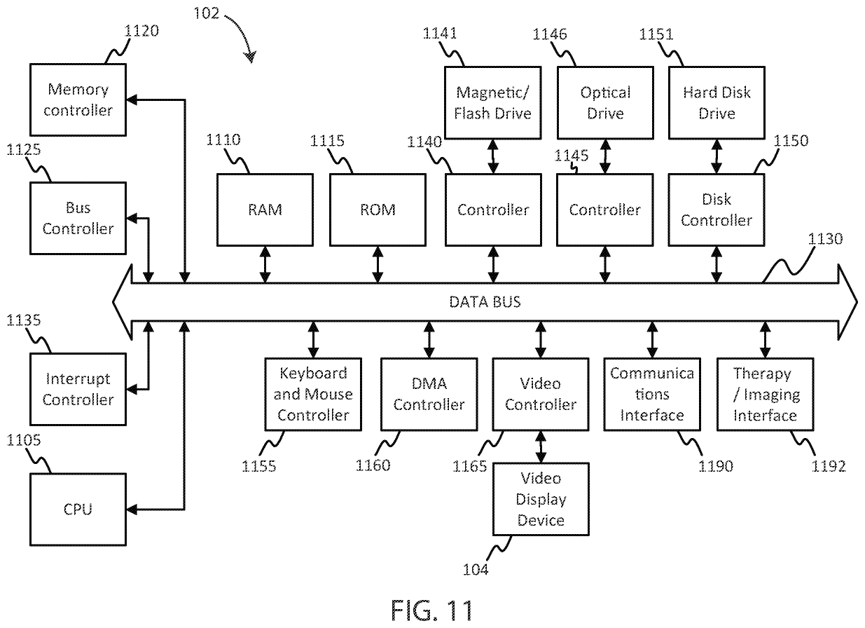

Systems and devices herein can include various components. Referring now to FIG. 11, a block diagram of components of a medical system 102 is shown in accordance with various embodiments herein. The medical visualization system can include a central processing circuit that can include various components such as a central processing unit. By way of example, the medical visualization system can include a central processing unit (CPU) 1105 or processor, which may include a conventional microprocessor, random access memory (RAM) 1110 for temporary storage of information, and read only memory (ROM) 1115 for permanent storage of information. A memory controller 1120 is provided for controlling system RAM 1110. A bus controller 1125 is provided for controlling data bus 1130, and an interrupt controller 1135 is used for receiving and processing various interrupt signals from the other system components.

Mass storage can be provided by a magnetic or flash memory drive 1141 including removable or non-removable media, which is connected to bus 1130 by controller 1140, an optical drive such as CD-ROM or DVD drive 1146, which is connected to bus 1130 by controller 1145, and/or hard disk drive 1151 (magnetic or solid state), which is connected to bus 1130 by controller 1150. In some embodiments, mass storage can be provided by a device connected through a universal serial bus (USB), eSATA, FireWire, or Thunderbolt interface or other type of connection. User input to the programmer system may be provided by a number of devices. For example, a keyboard and mouse can be connected to bus 1130 by keyboard and mouse controller 1155. DMA controller 1160 is provided for performing direct memory access to system RAM 1110. In some embodiments user input can be provided by a pen, light pen, glove, wearable object, gesture control interface, or the like.

A video processing circuit can be included and can generate a user interface. The video processing circuit can include a video controller 1165 or video output, which controls video display 104. In some embodiments, the video controller 1165 can also include one or more graphical processing units (GPUs). The video processing circuit can be in communication with the central processing circuit.

The medical system can also include a communications interface 1190 or communications circuit which allows the system to interface and exchange data with other systems and/or servers. The communications circuit can be in communication with the central processing circuit. In some embodiments, the communications interface 1190 can include a network interface card or circuit to facilitate communication with a packet switched (such as IP) or other type of data network.

The medical system can also include a therapy/imaging interface 1192. The therapy/imaging interface 1192 can be configured to facilitate the provision of therapy and/or execute tasks related to imaging.

It will be appreciated that some embodiments may lack various elements illustrated in FIG. 11. In addition, the architecture shown in FIG. 11 is merely one example of how discrete components can be arranged and other architectures are explicitly contemplated herein.

In addition to, or instead of, the components described with respect to FIG. 11, it will be appreciated that the system can also include a microcontroller, a programmable logic controller (PLC), an ASIC, an FPGA, a microprocessor, or other suitable technology. In various embodiments, one or more of the components shown in FIG. 11 and/or circuits serving the same or similar functions can serve as a control circuit herein.

The video processing circuit (either locally or on a remote node) can generate a 3D (or fewer or more dimensions) image based on information including one or more of geometry, viewpoint, texture, lighting and shading information, and other information described above. In some embodiments, information for rendering an image is combined within a scene file. The term “graphics pipeline” can be used to refer to the sequence of steps used to create a 2D raster representation of a 3D scene. The video processing circuit can execute one or more steps of the graphics pipeline. The video processing circuit can also include one or more physical components used in the graphics pipeline. Using the information described above, the graphics pipeline can include one or more stages of creating a scene out of geometric primitives, modelling and transformation, camera transformation, lighting, projection transformation, clipping, scan conversion or rasterization, and texturing and fragment shading. In various embodiments, other operations can also be performed. In various embodiments, the graphics pipeline can use OpenGL, DirectX, or other protocols.

### Methods

Many different methods are contemplated herein, including, but not limited to, methods of making, methods of using, and the like. Aspects of system/device operation described elsewhere herein can be performed as operations of one or more methods in accordance with various embodiments herein.

In various embodiments, operations described herein and method steps can be performed as part of a computer-implemented method executed by one or more processors of one or more computing devices. In various embodiments, operations described herein and method steps can be implemented instructions stored on a non-transitory, computer-readable medium that, when executed by one or more processors, cause a system to execute the operations and/or steps.

In an embodiment, a method of maintaining procedure data on a medical system open for use by unauthenticated users is included. The method can include hashing direct patient identifying data along with a salt value using a hash function to generate an identification string. The method can further include saving data regarding operations of medical system during procedure sessions and procedure timing as linked to identification strings.

In an embodiment, the method can further include discarding the salt value at the end of a procedure session.

In an embodiment, the method can further include receiving the direct patient identifying data from an unauthenticated user.

In an embodiment, the method can further include receiving inputs from an unauthenticated user regarding desired operations of the medical system.

In an embodiment, the method can further include presenting identification strings and procedure time information to a system user to verify the association of a procedure record to a particular patient.

### Hashing Algorithms

Various embodiments herein can include the executions of a hashing algorithm as part of an operation herein. Further details about the hashing algorithms are provided as follows. However, it will be appreciated that this is merely provided by way of example and that further variations are contemplated herein. In various embodiments, the generation of the identification string can be performed according to the following:

rID=pHash(pID,salt)

wherein rID is the generated identification string, pHash is the hashing algorithm used, pID is the direct patient identifying data, and salt is the salt value.

Various different types of hashing algorithms can be used. In many embodiments, a one-way hash function/algorithm can be used. In some embodiments, a password hash function/algorithm can be used. Generally, a password hash function/algorithm is a one-way function that is functionally slow, making brute force approaches difficult.

Hashing algorithms can include Message Digest (MDx) algorithms, such as MD5, and Secure Hash Algorithms (SHA), such as SHA-1, SHA-2 (such as SHA-256), and SHA-3 families, bcrypt, scrypt, Argon2, or the like. In some embodiments, the password hash function can specifically be Argon2.

Various parameters associated with some hashing algorithms can be set. For example, one or more of parallelism factor, memory, iterations, hash length, and the like can be set. In various embodiments herein, the hash length can be set to be greater than or equal to 2, 3, 4, 5, 6, 7, 8, 9, 10, 12, 15, 20, 25, 30, 40, 50 or more, or a length falling within a range between any of the foregoing.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A medical system comprising:
a control circuit;
a video display; and
a user interface, wherein the user interface is controlled by the control circuit and displayed on the video display;
wherein the medical system is configured to
permit system use by an unauthenticated user;
hash direct patient identifying data along with a salt value using a hash function to generate an identification string; and
save data regarding operations of the medical system during procedure sessions and procedure timing as linked to identification strings.

2. The medical system of claim 1, wherein the medical system is configured to discard the salt value at the end of a procedure session.

3. The medical system of claim 1, the salt value comprising a string of characters derived from a cryptographic random number.

4. The medical system of claim 1, the direct patient identifying data comprising a patient ID.

5. The medical system of claim 1, the data regarding operations comprising operational parameters.

6. The medical system of claim 1, the hash function comprising a password hash function.

7. The medical system of claim 6, the password hash function comprising Argon2.

8. The medical system of claim 1, the medical system comprising at least one selected from the group consisting of an Mill machine, an endoscopy device, a urological device, an RF generator, and a medical laser device.

9. The medical system of claim 1, wherein the medical system is configured to receive the direct patient identifying data from the unauthenticated user.

10. The medical system of claim 1, wherein the medical system is configured to receive inputs from the unauthenticated user regarding desired operations of the medical system.

11. The medical system of claim 1, wherein the medical system is configured to present identification strings and procedure time information to a system user to verify the association of a procedure record to a particular patient.

12. The medical system of claim 11, wherein the medical system prompts the system user to input direct patient identifying data.

13. The medical system of claim 11, wherein procedure records verified to be associated with a particular patient are stored in an electronic medical records system.

14. A method of maintaining procedure data on a medical system open for use by unauthenticated users comprising:
hashing direct patient identifying data along with a salt value using a hash function to generate an identification string; and
saving data regarding operations of medical system during procedure sessions and procedure timing as linked to identification strings.

15. The method of claim 14, further comprising discarding the salt value at the end of a procedure session.

16. The method of claim 14, further comprising receiving the direct patient identifying data from an unauthenticated user.

17. The method of claim 14, further comprising receiving inputs from an unauthenticated user regarding desired operations of the medical system.

18. The method of claim 14, further comprising presenting identification strings and procedure time information to a system user to verify the association of a procedure record to a particular patient.

19. The method of claim 14, the salt value comprising a string of characters derived from a cryptographic random number.

20. The method of claim 14, the direct patient identifying data comprising a patient ID.
